(19) United States
(12) Patent Application Publication
(10) Pub. No.: US 2020/0270614 A1
(43) Pub. Date: Aug. 27, 2020

(54) RNAi-BASED THERAPEUTICS FOR TARGETING HTRA1 AND METHODS OF USE

(71) Applicant: Gemini Therapeutics Inc., Cambridge, MA (US)

(72) Inventors: Walter Strapps, Cambridge, MA (US); James McLaughlin, Cambridge, MA (US)

(21) Appl. No.: 16/685,132

(22) Filed: Nov. 15, 2019

Related U.S. Application Data
(60) Provisional application No. 62/768,492, filed on Nov. 16, 2018.

(51) Int. Cl. C12N 15/113

(52) U.S. Cl. CPC C12N 15/1137; C12N 2310/11

(57) ABSTRACT

The present disclosure provides compositions and methods for treating, preventing, or inhibiting diseases of the eye. In one aspect, the disclosure provides HTRA1 RNAi agents and methods of using the same.

### CROSS-REFERENCE TO RELATED APPLICATIONS

This application claims the benefit of priority from U.S. Provisional Application No. 62/768,492, filed Nov. 16, 2018. The specification of the foregoing application is incorporated herein by reference in its entirety.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits. The size and number of drusen deposits typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision.

### SUMMARY OF THE DISCLOSURE

In some embodiments, the disclosure provides for an RNAi agent that targets an HTRA1 polynucleotide, wherein the HTRA1 polynucleotide encodes an HTRA1 polypeptide or functional fragment thereof. In some embodiments, the HTRA1 polypeptide comprises an amino acid sequence that is at least 80%, 85%, 90%, 93%, 95%, 97%, 98%, 99% or 100% identical to SEQ ID NO: 108. In some embodiments, the RNAi agent comprises a nucleotide sequence that is at least 80%, 85%, 90%, 93%, 95%, 97%, 98%, 99% or 100% identical to any one of SEQ ID NOs: 1-107. In some embodiments, the RNAi agent comprises the polynucleotide sequence of any of SEQ ID NOs: 1-107, but with 1, 2, 3, 4, 5, 6, 7, 8, 9, or 10 nucleotide modifications as compared to SEQ ID NOs: 1-107. In some embodiments, the RNAi agent comprises at least 8, 9, 10, 11, 12, 13, 14, 15, 16, 17, 18 or 19 contiguous nucleotides from a nucleotide sequence that is at least 80%, 85%, 90%, 93%, 95%, 97%, 98%, 99% or 100% identical to any one of SEQ ID NOs: 1-107. In some embodiments, the RNAi agent is capable of inhibiting the expression of an HTRA1 polypeptide. In some embodiments, the HTRA1 protein comprises an amino acid sequence that is at least 80%, 85%, 90%, 93%, 95%, 97%, 98%, 99% or 100% identical to SEQ ID NO: 108, or a functional fragment thereof. In some embodiments, the RNAi agent is capable of inhibiting the expression of a protein having an amino acid sequence that is at least 80%, 85%, 90%, 93%, 95%, 97%, 98%, 99% or 100% identical to SEQ ID NO: 108, or a functional fragment thereof. In some embodiments, the RNAi agent targets an mRNA transcript encoding the HTRA1 protein. In some embodiments, the mRNA transcript encoding the HTRA1 protein comprises a nucleotide sequence that is at least 80%, 85%, 90%, 93%, 95%, 97%, 98%, 99% or 100% identical to the nucleotide sequence of SEQ ID NO: 109 (but wherein thymines are replaced with uracil), or complements thereof. In some embodiments, the RNAi agent is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the RNAi agent. In some embodiments, the RNAi agent targets HTRA1-encoding mRNA for degradation. In some embodiments, the RNAi agent is capable of reducing HTRA1-encoding mRNA levels in a cell. In some embodiments, the RNAi agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the RNAi agent. In some embodiments, the RNAi agent comprises a sense and an antisense strand, wherein the sense and antisense strands contain the same number of nucleotides. In some embodiments, the RNAi agent comprises a sense and an antisense strand, wherein the sense and antisense strands contain a different number of nucleotides. In some embodiments, the RNAi agent comprises a sense and an antisense strand, wherein the sense strand 5′ end and the antisense strand 3′ end of an RNAi agent form a blunt end. In some embodiments, the RNAi agent comprises a sense and an antisense strand, wherein the sense strand 3′ end and the antisense strand 5′ end of an RNAi agent form a blunt end. In some embodiments, the RNAi agent comprises a sense and an antisense strand, wherein the sense strand 5′ end and the antisense strand 3′ end of an RNAi agent form a frayed end. In some embodiments, the RNAi agent comprises a sense and an antisense strand, wherein the sense strand 3′ end and the antisense strand 5′ end of an RNAi agent form a frayed end. In some embodiments, the RNAi agent comprises a sense and an antisense strand, wherein the RNAi agent comprises an overhang on the sense strand. In some embodiments, the RNAi agent comprises a sense and an antisense strand, wherein the RNAi agent comprises an overhang on the antisense strand. In some embodiments, the RNAi agent comprises one or more modified nucleotides. In some embodiments, the one or more modified nucleotides are selected from the group consisting of: deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, 2′-alkyl nucleotides, 5-substituted pyrimidines, 6-azapyriinidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine. In some embodiments, one or more nucleotides of the RNAi agent are linked by modified internucleoside linkages or backbones. In some embodiments, the modified internucleoside linkage or backbone is selected from the group consisting of: phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, amino alkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, boranophosphates having inverted polarity wherein the adjacent pairs of nucleoside units are linked 3′-5′ to 5′-3′ or 2′-5′ to 5′-2′, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH2 components. In some embodiments, the RNAi agent is a short interfering RNA (siRNA). In some embodiments, the RNAi agent is a double-strand RNA (dsRNA). In some embodiments, the RNAi agent is a micro RNA (miRNA). In some embodiments, the RNAi agent is a short hairpin RNA (shRNA). In some embodiments, the RNAi agent is a dicer substrate.

In some embodiments, the disclosure provides for a vector comprising any of the RNAi agents disclosed herein. In some embodiments, the vector is a viral vector. In some embodiments, the vector is an AAV vector. In some embodiments, the vector is a non-viral vector. In some embodiments, the vector is a nanoparticle. In some embodiments, the vector is a liposome.

In some embodiments, the disclosure provides for a host cell comprising any of the vectors disclosed herein.

In some embodiments, the disclosure provides for a method of treating a disease or disorder in a subject in need thereof, wherein the disease or disorder is associated with aberrantly expressed HTRA1, wherein the method comprises administering to the subject any of the RNAi agents or any of the vectors disclosed herein. In some embodiments, the disclosure provides a method of treating age-related macular degeneration or polypoidal choroidal vasculopathy in a subject in need thereof, wherein the method comprises administering to the subject any of the RNAi agents and/or any of the vectors disclosed herein. In some embodiments, the disclosure provides for a method of treating a disease or disorder in a subject in need thereof, wherein HTRA1 is expressed at a level at least 5%, 10%, 25%, 50%, 75%, 100%, 150%, 200%, 250%, 300%, 350%, 400%, 450%, or 500% greater in the subject having the disease or disorder as compared to the level in a control subject not having the disease or disorder, wherein the method comprises administering to the subject any of the RNAi agents or any of the vectors disclosed herein. In some embodiments, the control subject is a subject of the same sex and/or of similar age as the subject having the disease or disorder. In some embodiments, the subject has one or more mutations in the HTRA1 gene. In some embodiments, the one or more mutations are not in the coding sequence for the HTRA1 gene. In some embodiments, the one or more mutations are in 10q26 in a human subject. In some embodiments, the one or more mutations correspond to any one or more of the following polymorphisms in a human subject: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308. In some embodiments, the subject has age-related macular degeneration. In some embodiments, the subject is a human. In some embodiments, the human is at least 40 years of age. In some embodiments, the human is at least 50 years of age. In some embodiments, the human is at least 65 years of age. In some embodiments, the RNAi agent is administered locally. In some embodiments, the RNAi agent is administered intravitreally. In some embodiments, the RNAi agent is administered subretinally.

In some embodiments, the RNAi agent is administered systemically. In some embodiments, the subject has polypoidal choroidal vasculopathy. In some embodiments, the subject has Wet age-related macular degeneration. In some embodiments, the subject has Dry age-related macular degeneration.

In some embodiments, the disclosure provides for a composition comprising a pharmaceutically acceptable carrier and any of the RNAi agents disclosed herein and/or any of the vectors disclosed herein. In some embodiments, the composition is substantially pyrogen free.

### DETAILED DESCRIPTION OF THE DISCLOSURE

The disclosure provides compositions and methods for treating, preventing, or inhibiting diseases of the eye. In one aspect, the disclosure provides for HTRA1 RNAi agents. In another aspect, the disclosure provides methods of treating, preventing, or inhibiting diseases of the eye by intraocularly (e.g., intravitreally) administering an effective amount of any of the RNAi agents disclosed herein.

A wide variety of diseases of the eye may be treated or prevented using the RNAi agents and methods provided herein. Diseases of the eye that may be treated or prevented using the HTRA1 RNAi agents and methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Exemplary methods and materials are described herein, although methods and materials similar or equivalent to those described herein can also be used in the practice or testing of the present disclosure. The materials, methods, and examples are illustrative only and not intended to be limiting.

Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR', CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, a “base”, “nucleotide base,” or “nucleobase,” is a heterocyclic pyrimidine or purine compound, which is a standard constituent of all nucleic acids, and includes the bases that form the nucleotides adenine (A), guanine (G), cytosine (C), thymine (T), and uracil (U). A nucleobase may further be modified to include, without limitation, universal bases, hydrophobic bases, promiscuous bases, size-expanded bases, and fluorinated bases. As used herein, the term “nucleotide” can include a modified nucleotide (such as, for example, a nucleotide mimic, abasic residue (Ab), or a surrogate replacement moiety).

As used herein, the terms “sequence” and “nucleotide sequence” mean a succession or order of nucleobases or nucleotides, described with a succession of letters using standard nomenclature.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification.

As used herein, “perfectly complementary” or “fully complementary” means that all (100%) of the nucleobases or nucleotides in a contiguous sequence of a first polynucleotide will hybridize with the same number of nucleobases or nucleotides in a contiguous sequence of a second polynucleotide. The contiguous sequence may comprise all or a part of a first or second nucleotide sequence.

As used herein, “partially complementary” means that in a hybridized pair of nucleobase sequences, at least 70%, but not all, of the bases in a contiguous sequence of a first polynucleotide will hybridize with the same number of bases in a contiguous sequence of a second polynucleotide.

As used herein, “substantially complementary” means that in a hybridized pair of nucleobase sequences, at least 85%, but not all, of the bases in a contiguous sequence of a first polynucleotide will hybridize with the same number of bases in a contiguous sequence of a second polynucleotide. The terms “complementary,” “fully complementary,” “partially complementary,” and “substantially complementary” herein are used with respect to the nucleobase or nucleotide matching between the sense strand and the antisense strand of an RNAi agent, or between the antisense strand of an RNAi agent and a sequence of a target mRNA (e.g., an HTRA1 mRNA transcript).

As used herein, a “host cell” includes an individual cell or cell culture that can be or has been a recipient for vector(s) for incorporation of polynucleotide inserts. The term host cell may refer to the packaging cell line in which the RNAi agent is produced from the plasmid.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

In one embodiment, the subject has, or is at risk of developing a disease of the eye. A disease of the eye, includes, without limitation, AMD, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject is in need of optogenetic therapy. In another embodiment, the subject has shown clinical signs of a disease of the eye. In some embodiments, the subject has, or is at risk of developing AMD.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent”, “preventing” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent”, “preventing” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

As used herein, the term “capable of” means that the referenced composition (e.g., RNAi agent) has the capability to perform a specific function, but that it is not required to be performing that specific function at any specific moment in time. The term “capable of” encompasses instances where the composition is actively performing a specific function.

Each embodiment described herein may be used individually or in combination with any other embodiment described herein.

RNAi Agents

HTRA1 is a serine protease that targets a variety of proteins, including extracellular matrix proteins such as fibronectin. Fibronectin fragments resulting from HTRA1 cleavage are able to further induce synovial cells to up-regulate MMP1 and MMP3 production. There is evidence that HTRA1 may also degrade proteoglycans, such as aggrecan, decorin and fibromodulin. By cleaving proteoglycans, HTRA1 may release soluble FGF-glycosaminoglycan complexes that promote the range and intensity of FGF signals in the extracellular space. HTRA1 also regulates the availability of insulin-like growth factors (IGFs) by cleaving IGF-binding proteins. Intracellularly, HTRA1 degrades TSC2, leading to the activation of TSC2 downstream targets.

Overexpression of HTRA1 alters the integrity of Bruch's membrane, which permits choroid capillaries to invade across the extracellular matrix in conditions such as wet age-related macular degeneration. Tong et al., 2010, Mol. Vis., 16:1958-81. HTRA1 also inhibits signaling mediated by TGF-beta family members, which may regulate many physiological processes, including retinal angiogenesis and neuronal survival and maturation during development. It has been previously determined that a single-nucleotide polymorphism (rs11200638) in the promoter region of the HTRA1 gene was found to be significantly associated with susceptibility to AMD in various patient populations. Tong et al., 2010.

In some embodiments, any of HTRA1 RNAi agents disclosed herein are capable of decreasing proteolytic activity of an HTRA1 protein in a cell, tissue (e.g., eye) or organ. In some embodiments, the HTRA1 RNAi agent is capable of decreasing proteolytic activity by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the absence of the RNAi agent. In some embodiments, the RNAi agent is capable of reducing the HTRA1 proteolytic activity in a cell by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the same cell type in the absence of the RNAi agent. In some embodiments, the RNAi agent is capable of reducing HTRA1 proteolytic activity in an eye by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in an eye in the absence of the RNAi agent.

In some embodiments, the RNAi agent is capable of reducing HTRA1 cleavage of any one or more HTRA1 substrate. In some embodiments, the HTRA1 substrate is selected from the group consisting of: fibromodulin, clusterin, ADAMS, elastin, vitronectin, a2-macroglobulin, talin-1, fascin, LTBP-1, EFEMP1, and chloride intracellular channel protein. In some embodiments, the RNAi agent is capable of reducing HTRA1 cleavage of any one or more regulator of the complement cascade (e.g., vitronectin, fibromodulin or clusterin). In some embodiments, the RNAi agent is capable of reducing HTRA1 cleavage of any one or more HTRA1 substrate and/or regulator of the complement cascade by at least 5%, 10%, 15%, 25%, 30%, 35%, 40%, 45%, 50%, 55%, 60%, 65%, 70%, 75%, 80%, 85%, 90%, 95% or 100% as compared to the ability of the HTRA1 to cleave the HTRA1 substrate and/or regulator of the complement cascade in the absence of the RNAi agent.

As used herein, an “RNAi agent” or “RNAi agent” means an RNA or RNA-like (e.g., chemically modified RNA) oligonucleotide molecule that is capable of degrading or inhibiting translation of messenger RNA (mRNA) transcripts of a target mRNA (e.g., an HTRA1 mRNA transcript) in a sequence specific manner. In some embodiments, RNAi agents may operate through the RNA interference mechanism (i.e., inducing RNA interference through interaction with the RNA interference pathway machinery (RNA-induced silencing complex or RISC) of mammalian cells), or by any similar alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short interfering RNAs (siRNAs), double-strand RNAs (dsRNA), micro RNAs (miRNAs), short hairpin RNAs (shRNA), and dicer substrates. The antisense strand of the RNAi agents described herein is at least partially complementary to the mRNA being targeted (e.g. HTRA1 mRNA). RNAi agents can include one or more modified nucleotides and/or one or more non-phosphodiester linkages.

In some embodiments, any of the RNAi agents disclosed herein comprise a nucleotide sequence that is at least 80%, 85%, 90%, 93%, 95%, 97%, 98%, 99% or 100% identical to any one of SEQ ID NOs: 1-107. In some embodiments, any of the RNAi agents disclosed herein comprise any of the nucleotide sequences disclosed herein, but with at least one or more of any of the nucleotide modifications disclosed herein. In some embodiments, any of the RNAi agents disclosed herein comprises the polynucleotide sequence of any of SEQ ID NOs: 1-107, but with 1, 2, 3, 4, 5, 6, 7, 8, 9, or 10 nucleotide modifications as compared to the corresponding SEQ ID NO: 1-107. For example, an RNAi agent may comprise the nucleotide sequence of SEQ ID NO: 1, but with 2 nucleotide modifications as compared to SEQ ID NO: 1; or the RNAi agent may comprise the nucleotide sequence of SEQ ID NO: 2, but with 1 nucleotide modification as compared to SEQ ID NO: 2. In some embodiments, any of the RNAi agents disclosed herein comprises at least 8, 9, 10, 11, 12, 13, 14, 15, 16, 17, 18 or 19 contiguous nucleotides present from a nucleotide sequence that is at least 80%, 85%, 90%, 93%, 95%, 97%, 98%, 99% or 100% identical to any one of SEQ ID NOs: 1-107.

In some embodiments, any of the RNAi agents disclosed herein is capable of inhibiting the expression of an HTRA1 protein. In some embodiments, the HTRA1 protein comprises an amino acid sequence that is at least 80%, 85%, 90%, 93%, 95%, 97%, 98%, 99% or 100% identical to SEQ ID NO: 108, or a functional fragment thereof. In some embodiments, any of the RNAi agents disclosed herein is capable of inhibiting the expression of a protein having an amino acid sequence that is at least 80%, 85%, 90%, 93%, 95%, 97%, 98%, 99% or 100% identical to SEQ ID NO: 108, or a functional fragment thereof. In some embodiments, any of the RNAi agents disclosed herein target an mRNA transcript encoding the HTRA1 protein. In some embodiments, the mRNA transcript encoding the HTRA1 protein comprises a nucleotide sequence that is at least 80%, 85%, 90%, 93%, 95%, 97%, 98%, 99% or 100% identical to the nucleotide sequence of SEQ ID NO: 109 (but wherein thymines are replaced with uracil), or complements thereof. In some embodiments, any of the RNAi agents disclosed herein targets an mRNA transcript that is at least 80%, 85%, 90%, 93%, 95%, 97%, 98%, 99% or 100% identical to the nucleotide sequence of SEQ ID NO: 109 (but wherein thymines are replaced with uracil, or complements thereof). In some embodiments, any of the RNAi agents disclosed herein is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the RNAi agent. In some embodiments, the RNAi agent is capable of targeting the HTRA1-encoding mRNA for degradation. In some embodiments, any of the RNAi agents disclosed herein is capable of reducing HTRA1-encoding mRNA levels in a cell. In some embodiments, the RNAi agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the RNAi agent.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a blunt end refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands from a pair (i.e., do not form an overhang) but are not complementary (i.e. form a non-complementary pair). As used herein, an overhang is a stretch of one or more unpaired nucleotides at the end of one strand of a double stranded RNAi agent. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends.

Modified nucleotides, when used in various polynucleotide or oligonucleotide constructs, can preserve activity of the compound in cells while at the same time increasing the serum stability of these compounds, and can also minimize the possibility of activating interferon activity in humans upon administering of the polynucleotide or oligonucleotide construct.

In some embodiments, an HTRA1 RNAi agent is prepared or provided as a salt, mixed salt, or a free-acid. In some embodiments, an HTRA1 RNAi agent is prepared as a sodium salt. Such forms are within the scope of the inventions disclosed herein.

In some embodiments, an HTRA1 RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification can be incorporated in a single HTRA1 RNAi agent or even in a single nucleotide thereof. The HTRA1 RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyriinidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an RNAi agent is a ribonucleotide.

In some embodiments, one or more nucleotides of an HTRA1 RNAi agent are linked by non-standard linkages or backbones (i.e., modified internucleoside linkages or modified backbones).

Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, amino alkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH2 components.

In some embodiments, a sense strand of an HTRA1 RNAi agent can contain 1, 2, 3, 4, 5, or 6 phosphorothioate linkages, an antisense strand of an HTRA1 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an HTRA1 RNAi agent can contain 1, 2, 3, or 4 phosphorothioate linkages, an antisense strand of an HTRA1 RNAi agent can contain 1, 2, 3, or 4 phosphorothioate linkages, or both the sense strand and the antisense strand independently can contain 1, 2, 3, or 4 phosphorothioate linkages.

In some embodiments, an HTRA1 RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, an HTRA1 RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, an HTRA1 RNAi agent contains at least two phosphorothioate internucleoside linkages in the sense strand and three or four phosphorothioate internucleoside linkages in the antisense strand.

In some embodiments, any of the RNAi agents disclosed herein contains one or more modified nucleotides and one or more modified internucleoside linkages. In some embodiments, a 2′-modified nucleoside is combined with modified internucleoside linkage.

In some embodiments, any of the RNAi agents disclosed herein (e.g., an HTRA1 RNAi agent) is conjugated to one or more non-nucleotide groups including, but not limited to, a targeting group, linking group, delivery polymer, or a delivery vehicle. In some embodiments, the non-nucleotide group can enhance targeting, delivery or attachment of the RNAi agent. In some embodiments, the non-nucleotide group can be covalently linked to the 3′ and/or 5′ end of either the sense strand and/or the antisense strand. In some embodiments, an HTRA1 RNAi agent contains a non-nucleotide group linked to the 3′ and/or 5′ end of the sense strand. In some embodiments, a non-nucleotide group is linked to the 5′ end of an HTRA1 RNAi agent sense strand. In some embodiments, a non-nucleotide group can be linked directly or indirectly to the RNAi agent via a linker/linking group. In some embodiments, a non-nucleotide group is linked to the RNAi agent via a labile, cleavable, or reversible bond or linker. In some embodiments, a non-nucleotide group enhances the pharmacokinetic or biodistribution properties of an RNAi agent or conjugate to which it is attached to improve cell- or tissue-specific distribution and cell-specific uptake of the RNAi agent or conjugate. In some embodiments, a non-nucleotide group enhances endocytosis of the RNAi agent.

In some embodiments, a targeting group or targeting moiety can enhance the pharmacokinetic or biodistribution properties of a conjugate or RNAi agent to which they are attached to improve cell-specific distribution and cell-specific uptake of the conjugate or RNAi agent. In some embodiments, a targeting group can be monovalent, divalent, trivalent, tetravalent, or have higher valency for the target to which it is directed. In some embodiments, representative targeting groups include, without limitation, compounds with affinity to cell surface molecules, cell receptor ligands, haptens, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers. In some embodiments, a targeting group comprises a galactose derivative cluster.

In some embodiments, any of the HTRA1 RNAi agents described herein can be synthesized having a reactive group, such as an amine group, at the 5′-terminus. In some embodiments, the reactive group can be used to subsequently attach a targeting group using methods typical in the art.

In some embodiments, a linking group is conjugated to any of the RNAi agents disclosed herein. In some embodiments, the linking group facilitates covalent linkage of the agent to a targeting group or delivery polymer or delivery vehicle. In some embodiments, the linking group can be linked to the 3′ or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, can include, but are not limited to: reactive groups such a primary amines and alkynes, alkyl groups, abasic nucleotides, ribitol (abasic ribose), and/or PEG groups.

In some embodiments, a linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer can further add flexibility and/or length to the linkage. Spacers can include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides.

Spacer groups are well known in the art and the preceding list is not meant to limit the scope of the description.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. Suitable “moderately stringent conditions” include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2× SSC containing 0.1% SDS. As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC (sodium chloride/sodium citrate) and 50% formamide at 55° C., followed by a high-stringency wash consisting of 0.1× SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The nucleic acids/polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. In other embodiments, nucleic acids of the disclosure also include nucleotide sequences that hybridize under highly stringent conditions to the nucleotide sequences set forth in any of the sequences of SEQ ID NOs: 1-107 and 109, or sequences complementary thereto. One of ordinary skill in the art will readily understand that appropriate stringency conditions which promote DNA hybridization can be varied. For example, one could perform the hybridization at 6.0× sodium chloride/sodium citrate (SSC) at about 45° C., followed by a wash of 2.0× SSC at 50° C. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6× SSC at room temperature followed by a wash at 2× SSC at room temperature.

Isolated nucleic acids which differ due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. One skilled in the art will appreciate that these variations in one or more nucleotides (up to about 3-5% of the nucleotides) of the nucleic acids encoding a particular protein may exist among members of a given species due to natural allelic variation. Any and all such nucleotide variations and resulting amino acid polymorphisms are within the scope of this disclosure.

Pharmaceutical Compositions

Also provided herein are pharmaceutical compositions comprising an RNAi agent, and a pharmaceutically acceptable carrier. The pharmaceutical compositions may be suitable for any mode of administration described herein; for example, by intravitreal administration.

In some embodiments, use of any of the RNAi agents disclosed herein for treating retinal diseases, such as LCA, retinitis pigmentosa, and age-related macular degeneration require the localized delivery of the RNAi agent to the cells in the retina. In some embodiments, the cells that will be the treatment target in these diseases are either the photoreceptor cells in the retina or the cells of the RPE underlying the neurosensory retina.

In some embodiments, the pharmaceutical compositions comprising any of the RNAi agents described herein and a pharmaceutically acceptable carrier are suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising any of the RNAi agents described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, any of the RNAi agents disclosed herein is formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Routes of administration may be combined, if desired.

In some embodiments, any of the RNAi agents/pharmaceutical compositions disclosed herein are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if the RNAi agent/composition is to be administered to an elderly adult (e.g., at least 60 years of age). In particular embodiments, any of the RNAi agents/pharmaceutical compositions disclosed herein are administered to a subject intravitreally. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues {e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

The composition may be delivered in a volume of from about 0.1 μL to about 1 mL, including all numbers within the range, depending on the size of the area to be treated, the viral titer used (if the RNAi agent is administered using a viral vector), the route of administration, and the desired effect of the method. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

In some embodiments, any of the RNAi agents disclosed herein is administered via a vector. In some embodiments, the vector is a viral vector. In some embodiments, the viral vector is a retrovirus, lentivirus, or baculovirus vector. In some embodiments, the viral vector is an adenoviral vector. In particular embodiments, the viral vector is an AAV vector. A variety of rAAV vectors may be used to deliver the desired RNAi agent to the eye and to direct its expression. More than 30 naturally occurring serotypes of AAV from humans and non-human primates are known. Many natural variants of the AAV capsid exist, and an rAAV vector of the disclosure may be designed based on an AAV with properties specifically suited for ocular cells.

Recombinant AAV vectors of the present disclosure may be generated from a variety of adeno-associated viruses. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In certain embodiments, the AAV serotypes include AAVrh8, AAVrh8R or AAVrh10. It will also be understood that the rAAV vectors may be chimeras of two or more serotypes selected from serotypes AAV1 through AAV12. The tropism of the vector may be altered by packaging the recombinant genome of one serotype into capsids derived from another AAV serotype. In some embodiments, the ITRs of the rAAV virus may be based on the ITRs of any one of AAV1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In certain embodiments, the AAV capsid serotype is AAV2.

Desirable AAV fragments for assembly into vectors may include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique using a selected AAV sequence (e.g., a fragment of a vpl capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is incorporated by reference herein. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In some embodiments, the cap is derived from AAV2.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequences or a yeast ladder sequence. In some embodiments, the spacer is of a size sufficient to reduce expression of the rep78 and rep68 gene products, leaving the rep52, rep40 and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the capsid is modified to improve therapy. The capsid may be modified using conventional molecular biology techniques. In certain embodiments, the capsid is modified for minimized immunogenicity, better stability and particle lifetime, efficient degradation, and/or accurate delivery of the RNAi agent. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, any of the RNAi agents disclosed herein is administered to a cell, tissue, organ, or subject using a non-viral vector. In some embodiments, the non-viral vector is a cationic lipid- and/or polymer-based system. In some embodiments, the non-viral vector is a liposome or a nanoparticle. In some embodiments, any of the RNAi agents is administered to a cell, tissue, organ or subject by means of: sheddable ternary nanoparticles, lyophilized siRNA nanosome formulations, multicomponent synthetic polymers with viral-mimetic chemistry, scFv-mediated siRNA delivery, targeted polymeric micelles for siRNA, biscarbamate cross-linked low molecular weight PEI, carbonate apatite-mediated delivery, CADY/siRNA complexes, dendronized gold nanoparticles, glutathione-responsive nano-transporter, gelatin nanospheres, cationic lipbenzamides, lipid modified triblock PAMAM-based nanocarriers, bioresponsive and endosomolytic siRNA polymer conjugate, PAMAM dendrimer conjugates with cyclodextrins, GC-PEI nanoparticles, hemifluorinated polycationic lipids, PEI-based vector systems, glycopolymer-stabilized gold nanoparticles, siRNA nanogels and PCI, BBN-oligonucleotide conjugate, chlorotoxin bound magnetic nanovector, oral protein therapy, pH-sensitive carbonate apatite, amphoteric agmatine containing polyamidoamines, biodegradable dextran nanogels, dendrimer, poly(amine-co-esters), multilayered siRNA-coated gold nanoparticles, biodegradable amphiphilic and cationic triblock copolymer, siRNA/carbonate apatite nano-composites, polymeric vector incorporating endosomolytic oligomeric sulfonamide, lipid derivatives carrying amino and triazolyl groups, functional lipopolyamine, GPI modification, CADY self-assembling peptide-based nanoparticles, fluorescent PAMAM dendrimer, an injectable scaffold, amino-ethoxilated fluorinated amphiphile, and tyrosine trimers stabilized pDNA and siRNA polyplexes.

Methods of Treatment/Prophylaxis

Described herein are various methods of preventing, treating, arresting progression of or ameliorating the ocular disorders and retinal changes associated therewith. Generally, the methods include administering to a mammalian subject in need thereof, an effective amount of a composition comprising any of the RNAi agents disclosed herein. Any of the RNAi agents disclosed herein are useful in the methods described below.

In some embodiments, any of the RNAi agents disclosed herein are for use in treating retinal diseases, such as LCA, retinitis pigmentosa, and age-related macular degeneration may require the localized delivery of the RNAi agent to the cells in the retina. In some embodiments, the cells that will be the treatment target in these diseases are either the photoreceptor cells in the retina or the cells of the RPE underlying the neurosensory retina. In some embodiments, delivering any of the RNAi agents disclosed herein to these cells requires injection into the subretinal space between the retina and the RPE. In some embodiments, any of the RNAi agents disclosed herein are administered intravitreally or intravenously.

In a certain aspect, the disclosure provides a method of treating a subject having age-related macular degeneration (AMD), comprising the step of administering to the subject any of the RNAi agents of the disclosure. In some embodiments, the AMD is any one of Early AMD; Intermediate AMD; Advanced non-neovascular (“Dry”) AMD; or Advanced neovascular (“Wet”) AMD. In some embodiments, the disclosure provides for methods of treating a subject with Wet AMD. In some embodiments, the disclosure provides for methods of treating a subject with Dry AMD. In some embodiments, the disclosure provides for methods of treating a subject with polyploidal choroidal vasculopathy (PCV). In some embodiments, the subject has geographic atrophy.

In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic.

In some embodiments, any of HTRA1 RNAi agents disclosed results in a decrease of proteolytic activity of an HTRA1 protein in a cell, tissue, organ (e.g., eye) or subject. In some embodiments, the HTRA1 RNAi agent are capable of decreasing proteolytic activity by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the absence of the RNAi agent. In some embodiments, the RNAi agent is capable of decreasing HTRA1 proteolytic activity in a cell by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in the same cell type in the absence of the RNAi agent. In some embodiments, the RNAi agent is capable of reducing HTRA1 proteolytic activity in an eye by at least 5%, 10%, 15%, 25%, 30%, 35%, 40%, 45%, 50%, 55%, 60%, 65%, 70%, 75%, 80%, 85%, 90%, 95% or 100% as compared to the proteolytic activity of a wildtype HTRA1 protein in an eye in the absence of the RNAi agent.

In some embodiments, the RNAi agent is capable of reducing HTRA1 cleavage of any one or more HTRA1 substrate. In some embodiments, the HTRA1 substrate is selected from the group consisting of: fibromodulin, clusterin, elastin, ADAMS, vitronectin, a2-macroglobulin, talin-1, fascin, LTBP-1, EFEMPL and chloride intracellular channel protein. In some embodiments, the RNAi agent is capable of reducing HTRA1 cleavage of any one or more regulator of the complement cascade (e.g., vitronectin, fibromodulin or clusterin). In some embodiments, the RNAi agent is capable of reducing HTRA1 cleavage of any one or more HTRA1 substrate and/or regulator of the complement cascade by at least 5%, 10%, 15%, 25%, 30%, 35%, 40%, 45%, 50%, 55%, 60%, 65%, 70%, 75%, 80%, 85%, 90%, 95% or 100% as compared to the ability of the HTRA1 to cleave the HTRA1 substrate and/or regulator of the complement cascade in the absence of the RNAi agent.

In some embodiments, any of the RNAi agents disclosed herein is capable of inhibiting the expression of an HTRA1 protein in a cell, tissue, organ (e.g., eye) or subject. In some embodiments, the HTRA1 protein comprises an amino acid sequence that is at least 80%, 85%, 90%, 93%, 95%, 97%, 98%, 99% or 100% identical to SEQ ID NO: 108, or a functional fragment thereof. In some embodiments, any of the RNAi agents disclosed herein is capable of inhibiting the expression of a protein having an amino acid sequence that is at least 80%, 85%, 90%, 93%, 95%, 97%, 98%, 99% or 100% identical to SEQ ID NO: 108, or a functional fragment thereof. In some embodiments, any of the RNAi agents disclosed herein is capable of targeting an mRNA transcript encoding the HTRA1 protein. In some embodiments, the mRNA transcript encoding the HTRA1 protein comprises a nucleotide sequence that is at least 80%, 85%, 90%, 93%, 95%, 97%, 98%, 99% or 100% identical to the nucleotide sequence of SEQ ID NO: 109 (but wherein thymines are replaced with uracil), or complements thereof. In some embodiments, any of the RNAi agents disclosed herein is capable of targeting an mRNA transcript that is at least 80%, 85%, 90%, 93%, 95%, 97%, 98%, 99% or 100% identical to the nucleotide sequence of SEQ ID NO: 109 (but wherein thymines are replaced with uracil), or complements thereof. In some embodiments, any of the RNAi agents disclosed herein is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the RNAi agent. In some embodiments, the RNAi agent is capable of targeting the HTRA1-encoding mRNA for degradation. In some embodiments, any of the RNAi agents disclosed herein is capable of reducing HTRA1-encoding mRNA levels in a cell. In some embodiments, the RNAi agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the RNAi agent.

In some embodiments, any of the RNAi agents disclosed herein is administered to cell(s) or tissue(s) in a test subject. In some embodiments, the cell(s) or tissue(s) in the test subject express a higher level of HTRA1 than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye or a specific cell type of the eye (e.g., cells in the foveal region) in the test subject express at least 500%, 400%, 300%, 250%, 200%, 150%, 100%, 95%, 90%, 80%, 70%, 60%, 50%, 40%, 30%, 20%, 10%, 5%, or 1% more HTRA1 as compared to the levels in the reference control subject or population of reference control subjects. In some embodiments, the eye or a specific cell type of the eye (e.g., cells in the foveal region) in the test subject express an HTRA1 gene having any of the mutations disclosed herein. In some embodiments, the eye or a specific cell type of the eye (e.g., cells in the foveal region) in the reference control subject do not express a HTRA1 gene having any of the HTRA1 mutations disclosed herein. In some embodiments, administration of any of the RNAi agents disclosed herein in the cell(s) or tissue(s) of the test subject results in an decrease in levels of HTRA1 protein or functional HTRA1 protein. In some embodiments, administration of any of the RNAi agents disclosed herein in the cell(s) or tissue(s) of the test subject results in a decrease in levels of HTRA1 protein or functional HTRA1 protein such that the decreased levels are within 90%, 80%, 70%, 60%, 50%, 40%, 30%, 20%, 10%, 5%, or 1% of, or are the same as, the levels of HTRA1 protein or functional HTRA1 protein expressed by the same cell type or tissue type in the reference control subject or population of reference control subjects. In some embodiments, administration of any of the RNAi agents disclosed herein in the cell(s) or tissue(s) of the test subject results in a decrease in levels of HTRA1 protein or functional HTRA1 protein, but the decreased levels of HTRA1 protein or functional HTRA1 protein are not below the levels of HTRA1 protein or functional HTRA1 protein expressed by the same cell type or tissue type in the reference control subject or population of reference control subjects. In some embodiments, administration of any of the RNAi agents disclosed herein in the cell(s) or tissue(s) of the test subject results in a decrease in levels of HTRA1 protein or functional HTRA1 protein, but the decreased levels of HTRA1 protein or functional HTRA1 protein are below the levels of HTRA1 protein or functional HTRA1 protein by no more than 1%, 5%, 10%, 20%, 25%, 30%, 40%, 50%, 60%, 70%, 80%, 90% or 100% of the levels expressed by the same cell type or tissue type in the reference control subject or population of reference control subjects.

In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, 45, 50, 55, 60, 65, 70, 75, 80, 85, 90, or 95 years of age. In particular embodiments, the human is at least 60 or 65 years of age.

In some embodiments, any of the treatment and/or prophylactic methods disclosed herein is for use in treatment of a patient having one or more mutations that causes macular degeneration (AMD) or that increases the likelihood that a patient develops AMD. In some embodiments, the one or more mutations are in the patient's HTRA1 gene.

In some embodiments, any of the treatment and/or prophylactic methods disclosed herein is for use in treatment of a subject having one or more mutations in the patient's HTRA1 gene. As used herein, “mutations” encompasses polymorphisms that are associated with increased HTRA1 expression. In some embodiments, the one or more mutations result in overexpression of the HTRA1 gene. In some embodiments, HTRA1 is expressed at a level at least 25%, 50%, 75%, 100%, 150%, 200%, 250%, 300%, 350%, 400%, 450%, or 500% greater in the subject having the disease or disorder as compared to the level in a control subject not having the disease or disorder. In some embodiments, the control subject is a subject of the same sex and/or of similar age as the subject having the disease or disorder. In some embodiments, the one or more mutations are not in the coding sequence for the HTRA1 gene. In some embodiments, the one or more mutations are in 10q26 in a human patient. In some embodiments, the one or more mutations correspond to any one or more of the following human polymorphisms: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308.

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In another embodiment, a method of targeting one or more type(s) of ocular cells for gene augmentation therapy in a subject in need thereof is provided. In another embodiment, a method of targeting one or more type of ocular cells for gene suppression therapy in a subject in need thereof is provided. In yet another embodiment, a method of targeting one or more type of ocular cells for gene knockdown/augmentation therapy in a subject in need thereof is provided. In another embodiment, a method of targeting one or more type of ocular cells for gene correction therapy in a subject in need thereof is provided. In still another embodiment, a method of targeting one or more type of ocular cells for neurotropic factor gene therapy in a subject in need thereof is provided.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

In some embodiments, any of the methods disclosed herein increase photoreceptor function. As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod derived cone viability factor 2. The secondary therapy can be administered before, concurrent with, or after administration of the RNAi agents described above.

In some embodiments, any of the RNAi agents or compositions disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy.

Kits

In some embodiments, any of the RNAi agents disclosed herein is assembled into a pharmaceutical or diagnostic or research kit to facilitate their use in therapeutic, diagnostic or research applications. A kit may include one or more containers housing any of the RNAi agents disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

### EXAMPLES

The disclosure now being generally described, it will be more readily understood by reference to the following examples, which are included merely for purposes of illustration of certain embodiments and embodiments of the present disclosure, and are not intended to limit the disclosure.

### Example 1: Use of RNAi Agents for Treating AMD

This study will evaluate the efficacy of an RNAi agent comprising the nucleotide sequence of any one of SEQ ID NOs: 1-107 for treating patients with AMD. Patients with AMD will be treated with any of these RNAi agents, or a control. The RNAi agents will be administered at varying doses. The RNAi agents will be administered by intravitreal injection in a solution of PBS with additional NaCl and pluronic. Patients will be monitored for improvements in AMD symptoms.

It is expected that the RNAi treatments will improve the AMD symptoms.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

SEQUENCE LISTING

| | Sense Sequence |
|---|---|
| SEQ ID NO: 1 | AAAUCAAGGAUGUGGAUGA |
| SEQ ID NO: 2 | AAGCCAAAGAGCUGAAGGA |
| SEQ ID NO: 3 | AAGCCAUCACCAAGAAGAA |
| SEQ ID NO: 4 | ACAGUUUGCGCCAUAAAUA |
| SEQ ID NO: 5 | ACCGACAGGCCAAAGGAAA |
| SEQ ID NO: 6 | ACGCCAACACCUACGCCAA |
| SEQ ID NO: 7 | ACGGUGAAGUGAUUGGAAU |
| SEQ ID NO: 8 | ACUCAGACAUGGACUACAU |
| SEQ ID NO: 9 | AGGAAAACGACGUCAUAAU |
| SEQ ID NO: 10 | AGGAAGAUCCCAACAGUUU |
| SEQ ID NO: 11 | AGGAAGAUCCCAACAGUUU |
| SEQ ID NO: 12 | AGUGAUUCCCGAAGAAAUU |
| SEQ ID NO: 13 | AUGGACUGAUCGUGACAAA |
| SEQ ID NO: 14 | CAAAAUCAAGGAUGUGGAU |
| SEQ ID NO: 15 | CAACAAGCACCGGGUCAAA |
| SEQ ID NO: 16 | CAACAGUUUGCGCCAUAAA |
| SEQ ID NO: 17 | CAAGGAUGUGGAUGAGAAA |
| SEQ ID NO: 18 | CACCAAGAAGAAGUAUAUU |
| SEQ ID NO: 19 | CAGGAAGAUCCCAACAGUU |
| SEQ ID NO: 20 | CAGUGAUUCCCGAAGAAAU |
| SEQ ID NO: 21 | CCAAAAUCAAGGAUGUGGA |
| SEQ ID NO: 22 | CCAAAGAGCUGAAGGACCG |
| SEQ ID NO: 23 | CCAACAAGCACCGGGUCAA |
| SEQ ID NO: 24 | CCGAAGAAAUUGACCCAUA |
| SEQ ID NO: 25 | CCGACAGGCCAAAGGAAAA |
| SEQ ID NO: 26 | CCGCAGGGGUAAUGAAGAU |
| SEQ ID NO: 27 | CCUGGACGGUGAAGUGAUU |
| SEQ ID NO: 28 | CGGACGUGGUGGAGAAGAU |
| SEQ ID NO: 29 | CGGCCAAGGGCAGGAAGAU |
| SEQ ID NO: 30 | CGGUGAAGUGAUUGGAAUU |
| SEQ ID NO: 31 | CGUCAUAAUCAGCAUCAAU |
| SEQ ID NO: 32 | CGUGAUCUCAGGAGCGUAU |
| SEQ ID NO: 33 | CUGGUGGUCUCAAGGAAAA |
| SEQ ID NO: 34 | GAAAGUGACAGCUGGAAUC |
| SEQ ID NO: 35 | GAAGAAGUAUAUUGGUAUC |
| SEQ ID NO: 36 | GAAGAUGGACUGAUCGUGA |
| SEQ ID NO: 37 | GAGCGUAUAUAAUUGAAGU |
| SEQ ID NO: 38 | GCAAAGCCAAAGAGCUGAA |
| SEQ ID NO: 39 | GCAAAGCCAAAGAGCUGAA |
| SEQ ID NO: 40 | GCAACUCAGACAUGGACUA |
| SEQ ID NO: 41 | GCAGCGACGCCAACACCUA |
| SEQ ID NO: 42 | GCAGGAAGAUCCCAACAGU |
| SEQ ID NO: 43 | GCCCGUUAGUAAACCUGGA |
| SEQ ID NO: 44 | GCUAGUGGGUCUGGGUUUA |
| SEQ ID NO: 45 | GCUGGAAUCUCCUUUGCAA |
| SEQ ID NO: 46 | GCUGGUGGUCUCAAGGAAA |
| SEQ ID NO: 47 | GGAAAAGCCAUCACCAAGA |
| SEQ ID NO: 48 | GGAAGAUCCCAACAGUUUG |
| SEQ ID NO: 49 | GGCAGGAAGAUCCCAACAG |
| SEQ ID NO: 50 | GGCCAAGGGCAGGAAGAUC |
| SEQ ID NO: 51 | GGCUAGUGGGUCUGGGUUU |
| SEQ ID NO: 52 | GGGAAAGCACCCUGAACAU |
| SEQ ID NO: 53 | GGGCAGGAAGAUCCCAACA |
| SEQ ID NO: 54 | GGUGAAGUGAUUGGAAUUA |
| SEQ ID NO: 55 | GGUUUAUUGUGUCGGAAGA |
| SEQ ID NO: 56 | GUGAAGUGAUUGGAAUUAA |
| SEQ ID NO: 57 | UAGUAAACCUGGACGGUGA |
| SEQ ID NO: 58 | UCAAGGAUGUGGAUGAGAA |
| SEQ ID NO: 59 | UCACCAAGAAGAAGUAUAU |
| SEQ ID NO: 60 | UCACCAAGAAGAAGUAUAU |
| SEQ ID NO: 61 | UCGCGGACGUGGUGGAGAA |
| SEQ ID NO: 62 | UCUCAGGAGCGUAUAUAAU |
| SEQ ID NO: 63 | UGAAAGUGACAGCUGGAAU |
| SEQ ID NO: 64 | UGAAGAACGGUGCCACUUA |
| SEQ ID NO: 65 | GGCAGGAAGAUCCCAACAGUU |
| SEQ ID NO: 66 | GCAGGAAGAUCCCAACAGUUU |
| SEQ ID NO: 67 | GAUGGACUGAUCGUGACAAAU |
| SEQ ID NO: 68 | ACCAACAAGCACCGGGUCAAA |
| SEQ ID NO: 69 | AACAAGCACCGGGUCAAAGUU |
| SEQ ID NO: 70 | GCUGAAGAACGGUGCCACUUA |
| SEQ ID NO: 71 | AAAUCAAGGAUGUGGAUGAGA |
| SEQ ID NO: 72 | AAUCAAGGAUGUGGAUGAGAA |
| SEQ ID NO: 73 | AGAAAGCAGACAUCGCACUCA |
| SEQ ID NO: 74 | GAAAGCAGACAUCGCACUCAU |
| SEQ ID NO: 75 | AAGCAGACAUCGCACUCAUCA |
| SEQ ID NO: 76 | AGCAGACAUCGCACUCAUCAA |
| SEQ ID NO: 77 | GCAGACAUCGCACUCAUCAAA |
| SEQ ID NO: 78 | GCAACUCAGACAUGGACUACA |
| SEQ ID NO: 79 | AAACCUGGACGGUGAAGUGAU |
| SEQ ID NO: 80 | GGUGAAGUGAUUGGAAUUAAC |
| SEQ ID NO: 81 | GUGAAGUGAUUGGAAUUAACA |
| SEQ ID NO: 82 | GAAAGUGACAGCUGGAAUCUC |
| SEQ ID NO: 83 | AAGCCAUCACCAAGAAGAAGU |
| SEQ ID NO: 84 | AGCCAUCACCAAGAAGAAGUA |
| SEQ ID NO: 85 | AUUGGUAUCCGAAUGAUGUCA |
| SEQ ID NO: 86 | GUCCAGCAAAGCCAAAGAGCU |
| SEQ ID NO: 87 | GUGAUCUCAGGAGCGUAUAUA |
| SEQ ID NO: 88 | GAUCUCAGGAGCGUAUAUAAU |
| SEQ ID NO: 89 | AUCUCAGGAGCGUAUAUAAUU |
| SEQ ID NO: 90 | AGGAGCGUAUAUAAUUGAAGU |
| SEQ ID NO: 91 | GGAGCGUAUAUAAUUGAAGUA |
| SEQ ID NO: 92 | GACGUCAUAAUCAGCAUCAAU |
| SEQ ID NO: 93 | AGAGGCAUGAGCUGGACUUCA |
| SEQ ID NO: 94 | GCUGCUGGAAUAGGACACUCA |
| SEQ ID NO: 95 | GCUGGAAUAGGACACUCAAGA |
| SEQ ID NO: 96 | GGAAUAGGACACUCAAGACUU |
| SEQ ID NO: 97 | GCUCUGCCCUUCUGUAUCCUA |
| SEQ ID NO: 98 | GCCCUUCUGUAUCCUAUGUAU |
| SEQ ID NO: 99 | GGGCCAUUCUUGCUUAGACAG |
| SEQ ID NO: 100 | GGCCAUUCUUGCUUAGACAGU |
| SEQ ID NO: 101 | GCCAUUCUUGCUUAGACAGUC |
| SEQ ID NO: 102 | GUCUCCUCCUUUAACUGAGUC |
| SEQ ID NO: 103 | AGUCGAUACAAUGCGUAGAUA |
| SEQ ID NO: 104 | GUCGAUACAAUGCGUAGAUAG |
| SEQ ID NO: 105 | GGAAUUGGGAGCACGAUGACU |
| SEQ ID NO: 106 | GAAUUGGGAGCACGAUGACUC |
| SEQ ID NO: 107 | AAUUGGGAGCACGAUGACUCU |

Human HTRA1 Amino Acid Sequence- GenBank
Accession No. NP_002766.1
SEQ ID NO: 108
MQIPRAALLPLLLLLLAAPASAQLSRAGRSAPLAAGCPDRCEPARCPP
QPEHCEGGRARDACGCCEVCGAPEGAACGLQEGPCGEGLQCVVPFGVP
ASATVRRRAQAGLCVCASSEPVCGSDANTYANLCQLRAASRRSERLHR
PPVIVLQRGACGQGQEDPNSLRHKYNFIADVVEKIAPAVVHIELFRKL
PFSKREVPVASGSGFIVSEDGLIVTNAHVVTNKHRVKVELKNGATYEA
KIKDVDEKADIALIKIDHQGKLPVLLLGRSSELRPGEFVVAIGSPFSL
QNTVTTGIVSTTQRGGKELGLRNSDMDYIQTDAIINYGNSGGPLVNLD
GEVIGINTLKVTAGISFAIPSDKIKKFLTESHDRQAKGKAITKKKYIG
IRMMSLTSSKAKELKDRHRDFPDVISGAYIIEVIPDTPAEAGGLKEND
VIISINGQSVVSANDVSDVIKRESTLNMVVRRGNEDIMITVIPEEIDP
Human HTRA1 Polynucleotide Sequence- GenBank
Accession No. NM_002775.4
SEQ ID NO: 109
CAATGGGCTGGGCCGCGCGGCCGCGCGCACTCGCACCCGCTGCCCCCG
AGGCCCTCCTGCACTCTCCCCGGCGCCGCTCTCCGGCCCTCGCCCTGT
CCGCCGCCACCGCCGCCGCCGCCAGAGTCGCCATGCAGATCCCGCGCG
CCGCTCTTCTCCCGCTGCTGCTGCTGCTGCTGGCGGCGCCCGCCTCGG
CGCAGCTGTCCCGGGCCGGCCGCTCGGCGCCTTTGGCCGCCGGGTGCC
CAGACCGCTGCGAGCCGGCGCGCTGCCCGCCGCAGCCGGAGCACTGCG
AGGGCGGCCGGGCCCGGGACGCGTGCGGCTGCTGCGAGGTGTGCGGCG
CGCCCGAGGGCGCCGCGTGCGGCCTGCAGGAGGGCCCGTGCGGCGAGG
GGCTGCAGTGCGTGGTGCCCTTCGGGGTGCCAGCCTCGGCCACGGTGC
GGCGGCGCGCGCAGGCCGGCCTCTGTGTGTGCGCCAGCAGCGAGCCGG
TGTGCGGCAGCGACGCCAACACCTACGCCAACCTGTGCCAGCTGCGCG
CCGCCAGCCGCCGCTCCGAGAGGCTGCACCGGCCGCCGGTCATCGTCC
TGCAGCGCGGAGCCTGCGGCCAAGGGCAGGAAGATCCCAACAGTTTGC
GCCATAAATATAACTTTATCGCGGACGTGGTGGAGAAGATCGCCCCTG
CCGTGGTTCATATCGAATTGTTTCGCAAGCTTCCGTTTTCTAAACGAG
AGGTGCCGGTGGCTAGTGGGTCTGGGTTTATTGTGTCGGAAGATGGAC
TGATCGTGACAAATGCCCACGTGGTGACCAACAAGCACCGGGTCAAAG
TTGAGCTGAAGAACGGTGCCACTTACGAAGCCAAAATCAAGGATGTGG
ATGAGAAAGCAGACATCGCACTCATCAAAATTGACCACCAGGGCAAGC
TGCCTGTCCTGCTGCTTGGCCGCTCCTCAGAGCTGCGGCCGGGAGAGT
TCGTGGTCGCCATCGGAAGCCCGTTTTCCCTTCAAAACACAGTCACCA
CCGGGATCGTGAGCACCACCCAGCGAGGCGGCAAAGAGCTGGGGCTCC
GCAACTCAGACATGGACTACATCCAGACCGACGCCATCATCAACTATG
GAAACTCGGGAGGCCCGTTAGTAAACCTGGACGGTGAAGTGATTGGAA
TTAACACTTTGAAAGTGACAGCTGGAATCTCCTTTGCAATCCCATCTG
ATAAGATTAAAAAGTTCCTCACGGAGTCCCATGACCGACAGGCCAAAG
GAAAAGCCATCACCAAGAAGAAGTATATTGGTATCCGAATGATGTCAC
TCACGTCCAGCAAAGCCAAAGAGCTGAAGGACCGGCACCGGGACTTCC
CAGACGTGATCTCAGGAGCGTATATAATTGAAGTAATTCCTGATACCC
CAGCAGAAGCTGGTGGTCTCAAGGAAAACGACGTCATAATCAGCATCA
ATGGACAGTCCGTGGTCTCCGCCAATGATGTCAGCGACGTCATTAAAA
GGGAAAGCACCCTGAACATGGTGGTCCGCAGGGGTAATGAAGATATCA
TGATCACAGTGATTCCCGAAGAAATTGACCCATAGGCAGAGGCATGAG
CTGGACTTCATGTTTCCCTCAAAGACTCTCCCGTGGATGACGGATGAG
GACTCTGGGCTGCTGGAATAGGACACTCAAGACTTTTGACTGCCATTT
TGTTTGTTCAGTGGAGACTCCCTGGCCAACAGAATCCTTCTTGATAGT
TTGCAGGCAAAACAAATGTAATGTTGCAGATCCGCAGGCAGAAGCTCT
GCCCTTCTGTATCCTATGTATGCAGTGTGCTTTTTCTTGCCAGCTTGG
GCCATTCTTGCTTAGACAGTCAGCATTTGTCTCCTCCTTTAACTGAGT
CATCATCTTAGTCCAACTAATGCAGTCGATACAATGCGTAGATAGAAG
AAGCCCCACGGGAGCCAGGATGGGACTGGTCGTGTTTGTGCTTTTCTC
CAAGTCAGCACCCAAAGGTCAATGCACAGAGACCCCGGGTGGGTGAGC
GCTGGCTTCTCAAACGGCCGAAGTTGCCTCTTTTAGGAATCTCTTTGG
AATTGGGAGCACGATGACTCTGAGTTTGAGCTATTAAAGTACTTCTTA
CACATTGCAAAAAAAAAAAAAAAAAA

1. An RNAi agent that targets an HTRA1 polynucleotide, wherein the HTRA1 polynucleotide encodes an HTRA1 polypeptide or functional fragment thereof, and wherein the RNAi agent comprises a nucleotide sequence that is at least 80%, 85%, 90%, 93%, 95%, 97%, 98%, 99% or 100% identical to any one of SEQ ID NOs: 1-107.

2-4. (canceled)

5. The RNAi agent of claim 1, wherein the RNAi agent comprises at least 8, 9, 10, 11, 12, 13, 14, 15, 16, 17, 18 or 19 contiguous nucleotides from a nucleotide sequence of any one of SEQ ID NOs: 1-107.

6. The RNAi agent of claim 1, wherein the RNAi agent is capable of inhibiting the expression of an HTRA1 polypeptide.

7-11. (canceled)

12. The RNAi agent of claim 1, wherein the RNAi agent targets HTRA1-encoding mRNA for degradation.

13. (canceled)

14. The RNAi agent of claim 1, wherein the RNAi agent is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the RNAi agent.

15. The RNAi agent of claim 1, wherein the RNAi agent comprises a sense and an antisense strand, wherein the sense and antisense strands contain the same number of nucleotides.

16. The RNAi agent of claim 1, wherein the RNAi agent comprises a sense and an antisense strand, wherein the sense and antisense strands contain a different number of nucleotides.

17. The RNAi agent of claim 1, wherein the RNAi agent comprises a sense and an antisense strand, wherein the sense strand 5′ end and the antisense strand 3′ end of an RNAi agent form a blunt end or a frayed end.

18. The RNAi agent of claim 1, wherein the RNAi agent comprises a sense and an antisense strand, wherein the sense strand 3′ end and the antisense strand 5′ end of an RNAi agent form a blunt end or a frayed end.

19-20. (canceled)

21. The RNAi agent of claim 1, wherein the RNAi agent comprises a sense and an antisense strand, wherein the RNAi agent comprises an overhang on the sense strand and/or the antisense strand.

22. (canceled)

23. The RNAi agent of claim 1, wherein the RNAi agent comprises one or more modified nucleotides.

24. (canceled)

25. The RNAi agent of claim 1, wherein one or more nucleotides of the RNAi agent are linked by modified internucleoside linkages or backbones.

26. (canceled)

27. The RNAi agent of claim 1, wherein the RNAi agent is a short interfering RNA (siRNA), a double-strand RNA (dsRNA), a micro RNA (miRNA), a short hairpin RNA (shRNA), or a dicer substrate.

28-31. (canceled)

32. A vector comprising the RNAi agent of claim 1.

33-37. (canceled)

38. A host cell comprising the vector of claim 32.

39. A method of treating a disease or disorder in a subject in need thereof, wherein the disease or disorder is associated with aberrantly expressed HTRA1, wherein the method comprises administering to the subject the RNAi agent of claim 1 or a vector comprising the RNAi agent.

40. A method of treating age-related macular degeneration or polypoidal choroidal vasculopathy, wherein the method comprises administering to the subject the RNAi agent of claim 1 or a vector comprising the RNAi agent.

41. A method of treating a disease or disorder in a subject in need thereof, wherein HTRA1 is expressed at a level at least 5%, 10%, 25%, 50%, 75%, 100%, 150%, 200%, 250%, 300%, 350%, 400%, 450%, or 500% greater in the subject having the disease or disorder as compared to the level in a control subject not having the disease or disorder, wherein the method comprises administering to the subject the RNAi agent of claim 1 or a vector comprising the RNAi agent.

42-58. (canceled)

59. A composition comprising a pharmaceutically acceptable carrier and (i) the RNAi agent of claim 1 or (ii) a vector comprising the RNAi agent.

60. The composition of claim 59, wherein the composition is substantially pyrogen free.


SEQUENCE LISTING (109 sequences)

SEQ ID NO: 1 - RNA, 19 nt, Homo sapiens
aaaucaaggauguggauga

SEQ ID NO: 2 - RNA, 19 nt, Homo sapiens
aagccaaagagcugaagga

SEQ ID NO: 3 - RNA, 19 nt, Homo sapiens
aagccaucaccaagaagaa

SEQ ID NO: 4 - RNA, 19 nt, Homo sapiens
acaguuugcgccauaaaua

SEQ ID NO: 5 - RNA, 19 nt, Homo sapiens
accgacaggccaaaggaaa

SEQ ID NO: 6 - RNA, 19 nt, Homo sapiens
acgccaacaccuacgccaa

SEQ ID NO: 7 - RNA, 19 nt, Homo sapiens
acggugaagugauuggaau

SEQ ID NO: 8 - RNA, 19 nt, Homo sapiens
acucagacauggacuacau

SEQ ID NO: 9 - RNA, 19 nt, Homo sapiens
aggaaaacgacgucauaau

SEQ ID NO: 10 - RNA, 19 nt, Homo sapiens
aggaagaucccaacaguuu

SEQ ID NO: 11 - RNA, 19 nt, Homo sapiens
aggaagaucccaacaguuu

SEQ ID NO: 12 - RNA, 19 nt, Homo sapiens
agugauucccgaagaaauu

SEQ ID NO: 13 - RNA, 19 nt, Homo sapiens
auggacugaucgugacaaa

SEQ ID NO: 14 - RNA, 19 nt, Homo sapiens
caaaaucaaggauguggau

SEQ ID NO: 15 - RNA, 19 nt, Homo sapiens
caacaagcaccgggucaaa

SEQ ID NO: 16 - RNA, 19 nt, Homo sapiens
caacaguuugcgccauaaa

SEQ ID NO: 17 - RNA, 19 nt, Homo sapiens
caaggauguggaugagaaa

SEQ ID NO: 18 - RNA, 19 nt, Homo sapiens
caccaagaagaaguauauu

SEQ ID NO: 19 - RNA, 19 nt, Homo sapiens
caggaagaucccaacaguu

SEQ ID NO: 20 - RNA, 19 nt, Homo sapiens
cagugauucccgaagaaau

SEQ ID NO: 21 - RNA, 19 nt, Homo sapiens
ccaaaaucaaggaugugga

SEQ ID NO: 22 - RNA, 19 nt, Homo sapiens
ccaaagagcugaaggaccg

SEQ ID NO: 23 - RNA, 19 nt, Homo sapiens
ccaacaagcaccgggucaa

SEQ ID NO: 24 - RNA, 19 nt, Homo sapiens
ccgaagaaauugacccaua

SEQ ID NO: 25 - RNA, 19 nt, Homo sapiens
ccgacaggccaaaggaaaa

SEQ ID NO: 26 - RNA, 19 nt, Homo sapiens
ccgcagggguaaugaagau

SEQ ID NO: 27 - RNA, 19 nt, Homo sapiens
ccuggacggugaagugauu

SEQ ID NO: 28 - RNA, 19 nt, Homo sapiens
cggacgugguggagaagau

SEQ ID NO: 29 - RNA, 19 nt, Homo sapiens
cggccaagggcaggaagau

SEQ ID NO: 30 - RNA, 19 nt, Homo sapiens
cggugaagugauuggaauu

SEQ ID NO: 31 - RNA, 19 nt, Homo sapiens
cgucauaaucagcaucaau

SEQ ID NO: 32 - RNA, 19 nt, Homo sapiens
cgugaucucaggagcguau

SEQ ID NO: 33 - RNA, 19 nt, Homo sapiens
cugguggucucaaggaaaa

SEQ ID NO: 34 - RNA, 19 nt, Homo sapiens
gaaagugacagcuggaauc

SEQ ID NO: 35 - RNA, 19 nt, Homo sapiens
gaagaaguauauugguauc

SEQ ID NO: 36 - RNA, 19 nt, Homo sapiens
gaagauggacugaucguga

SEQ ID NO: 37 - RNA, 19 nt, Homo sapiens
gagcguauauaauugaagu

SEQ ID NO: 38 - RNA, 19 nt, Homo sapiens
gcaaagccaaagagcugaa

SEQ ID NO: 39 - RNA, 19 nt, Homo sapiens
gcaaagccaaagagcugaa

SEQ ID NO: 40 - RNA, 19 nt, Homo sapiens
gcaacucagacauggacua

SEQ ID NO: 41 - RNA, 19 nt, Homo sapiens
gcagcgacgccaacaccua

SEQ ID NO: 42 - RNA, 19 nt, Homo sapiens
gcaggaagaucccaacagu

SEQ ID NO: 43 - RNA, 19 nt, Homo sapiens
gcccguuaguaaaccugga

SEQ ID NO: 44 - RNA, 19 nt, Homo sapiens
gcuagugggucuggguuua

SEQ ID NO: 45 - RNA, 19 nt, Homo sapiens
gcuggaaucuccuuugcaa

SEQ ID NO: 46 - RNA, 19 nt, Homo sapiens
gcugguggucucaaggaaa

SEQ ID NO: 47 - RNA, 19 nt, Homo sapiens
ggaaaagccaucaccaaga

SEQ ID NO: 48 - RNA, 19 nt, Homo sapiens
ggaagaucccaacaguuug

SEQ ID NO: 49 - RNA, 19 nt, Homo sapiens
ggcaggaagaucccaacag

SEQ ID NO: 50 - RNA, 19 nt, Homo sapiens
ggccaagggcaggaagauc

SEQ ID NO: 51 - RNA, 19 nt, Homo sapiens
ggcuagugggucuggguuu

SEQ ID NO: 52 - RNA, 19 nt, Homo sapiens
gggaaagcacccugaacau

SEQ ID NO: 53 - RNA, 19 nt, Homo sapiens
gggcaggaagaucccaaca

SEQ ID NO: 54 - RNA, 19 nt, Homo sapiens
ggugaagugauuggaauua

SEQ ID NO: 55 - RNA, 19 nt, Homo sapiens
gguuuauugugucggaaga

SEQ ID NO: 56 - RNA, 19 nt, Homo sapiens
gugaagugauuggaauuaa

SEQ ID NO: 57 - RNA, 19 nt, Homo sapiens
uaguaaaccuggacgguga

SEQ ID NO: 58 - RNA, 19 nt, Homo sapiens
ucaaggauguggaugagaa

SEQ ID NO: 59 - RNA, 19 nt, Homo sapiens
ucaccaagaagaaguauau

SEQ ID NO: 60 - RNA, 19 nt, Homo sapiens
ucaccaagaagaaguauau

SEQ ID NO: 61 - RNA, 19 nt, Homo sapiens
ucgcggacgugguggagaa

SEQ ID NO: 62 - RNA, 19 nt, Homo sapiens
ucucaggagcguauauaau

SEQ ID NO: 63 - RNA, 19 nt, Homo sapiens
ugaaagugacagcuggaau

SEQ ID NO: 64 - RNA, 19 nt, Homo sapiens
ugaagaacggugccacuua

SEQ ID NO: 65 - RNA, 21 nt, Homo sapiens
ggcaggaagaucccaacaguu

SEQ ID NO: 66 - RNA, 21 nt, Homo sapiens
gcaggaagaucccaacaguuu

SEQ ID NO: 67 - RNA, 21 nt, Homo sapiens
gauggacugaucgugacaaau

SEQ ID NO: 68 - RNA, 21 nt, Homo sapiens
accaacaagcaccgggucaaa

SEQ ID NO: 69 - RNA, 21 nt, Homo sapiens
aacaagcaccgggucaaaguu

SEQ ID NO: 70 - RNA, 21 nt, Homo sapiens
gcugaagaacggugccacuua

SEQ ID NO: 71 - RNA, 21 nt, Homo sapiens
aaaucaaggauguggaugaga

SEQ ID NO: 72 - RNA, 21 nt, Homo sapiens
aaucaaggauguggaugagaa

SEQ ID NO: 73 - RNA, 21 nt, Homo sapiens
agaaagcagacaucgcacuca

SEQ ID NO: 74 - RNA, 21 nt, Homo sapiens
gaaagcagacaucgcacucau

SEQ ID NO: 75 - RNA, 21 nt, Homo sapiens
aagcagacaucgcacucauca

SEQ ID NO: 76 - RNA, 21 nt, Homo sapiens
agcagacaucgcacucaucaa

SEQ ID NO: 77 - RNA, 21 nt, Homo sapiens
gcagacaucgcacucaucaaa

SEQ ID NO: 78 - RNA, 21 nt, Homo sapiens
gcaacucagacauggacuaca

SEQ ID NO: 79 - RNA, 21 nt, Homo sapiens
aaaccuggacggugaagugau

SEQ ID NO: 80 - RNA, 21 nt, Homo sapiens
ggugaagugauuggaauuaac

SEQ ID NO: 81 - RNA, 21 nt, Homo sapiens
gugaagugauuggaauuaaca

SEQ ID NO: 82 - RNA, 21 nt, Homo sapiens
gaaagugacagcuggaaucuc

SEQ ID NO: 83 - RNA, 21 nt, Homo sapiens
aagccaucaccaagaagaagu

SEQ ID NO: 84 - RNA, 21 nt, Homo sapiens
agccaucaccaagaagaagua

SEQ ID NO: 85 - RNA, 21 nt, Homo sapiens
auugguauccgaaugauguca

SEQ ID NO: 86 - RNA, 21 nt, Homo sapiens
guccagcaaagccaaagagcu

SEQ ID NO: 87 - RNA, 21 nt, Homo sapiens
gugaucucaggagcguauaua

SEQ ID NO: 88 - RNA, 21 nt, Homo sapiens
gaucucaggagcguauauaau

SEQ ID NO: 89 - RNA, 21 nt, Homo sapiens
aucucaggagcguauauaauu

SEQ ID NO: 90 - RNA, 21 nt, Homo sapiens
aggagcguauauaauugaagu

SEQ ID NO: 91 - RNA, 21 nt, Homo sapiens
ggagcguauauaauugaagua

SEQ ID NO: 92 - RNA, 21 nt, Homo sapiens
gacgucauaaucagcaucaau

SEQ ID NO: 93 - RNA, 21 nt, Homo sapiens
agaggcaugagcuggacuuca

SEQ ID NO: 94 - RNA, 21 nt, Homo sapiens
gcugcuggaauaggacacuca

SEQ ID NO: 95 - RNA, 21 nt, Homo sapiens
gcuggaauaggacacucaaga

SEQ ID NO: 96 - RNA, 21 nt, Homo sapiens
ggaauaggacacucaagacuu

SEQ ID NO: 97 - RNA, 21 nt, Homo sapiens
gcucugcccuucuguauccua

SEQ ID NO: 98 - RNA, 21 nt, Homo sapiens
gcccuucuguauccuauguau

SEQ ID NO: 99 - RNA, 21 nt, Homo sapiens
gggccauucuugcuuagacag

SEQ ID NO: 100 - RNA, 21 nt, Homo sapiens
ggccauucuugcuuagacagu

SEQ ID NO: 101 - RNA, 21 nt, Homo sapiens
gccauucuugcuuagacaguc

SEQ ID NO: 102 - RNA, 21 nt, Homo sapiens
gucuccuccuuuaacugaguc

SEQ ID NO: 103 - RNA, 21 nt, Homo sapiens
agucgauacaaugcguagaua

SEQ ID NO: 104 - RNA, 21 nt, Homo sapiens
gucgauacaaugcguagauag

SEQ ID NO: 105 - RNA, 21 nt, Homo sapiens
ggaauugggagcacgaugacu

SEQ ID NO: 106 - RNA, 21 nt, Homo sapiens
gaauugggagcacgaugacuc

SEQ ID NO: 107 - RNA, 21 nt, Homo sapiens
aauugggagcacgaugacucu

SEQ ID NO: 108 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 109 - DNA, 2138 bp, Homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgcaaaaaaaaaaaaaaaaaa